(19) United States
(12) Patent Application Publication
(10) Pub. No.: US 2026/0133186 A1
(43) Pub. Date: May 14, 2026

(54) Tissue Imaging Replicas

(71) Applicant: Standard BioTools Canada Inc., Markham (CA)

(72) Inventor: Alexander LOBODA, Markham (CA)

(73) Assignee: Standard BioTools Canada Inc., Markham, ON (CA)

(21) Appl. No.: 19/119,034

(22) PCT Filed: Oct. 23, 2023
(86) PCT No.: PCT/IB2023/060690

§ 371 Date: Apr. 7, 2025

Related U.S. Application Data
(60) Provisional application No. 63/418,379, filed on Oct. 21, 2022.

(51) Int. Cl. G01N 33/543; G01N 1/30; G01N 33/50

(52) U.S. Cl. CPC G01N 33/54306; G01N 1/30; G01N 33/5005; G01N 2474/00

(57) ABSTRACT

A method of imaging a biological sample is disclosed, which comprises coupling at least one affinity probe to the biological sample, wherein the affinity probe includes a cleavable tag, bringing the probe-coupled biological sample into contact with a non-selective capture surface so as to couple said at least one cleavable tag to the capture surface, subsequently, cleaving the at least one tag from the affinity probe so as to transfer the at least one tag to the capture surface, thereby generating a replica of the biological sample.

### RELATED APPLICATIONS

This application claims priority to U.S. Provisional Application No. 63/418,379 filed on Oct. 21, 2022, the contents of which are incorporated herein in their entirety.

### TECHNICAL FIELD

The present disclosure is related to methods for imaging biological samples by generating replicas of such samples and imaging or probing the replicas, and use of replicas for single cell analysis.

### BACKGROUND

The present disclosure is generally directed to methods for imaging or probing of biological samples (e.g., tissues, cell pellets, cell smears, among others), and more particularly to such methods that can be used to generate replicas of biological samples of interest, which can be imaged or probed.

Analysis of biological samples, e.g., via imaging of the samples, is routinely employed in medical/biological research as well as for diagnostic purposes. For example, such analysis of a tissue section may be used for the quantitative detection of cell protein markers. In many cases, highly-multiplexed technologies are needed to analyze biomolecules such as polynucleotides and polypeptides contents of tissue sections or individual cells.

Conventional techniques for performing such analysis, however, pose certain shortcomings. Immunohistochemistry (IHC)-based techniques typically do not provide the capability for multiplexed analysis of a large number of proteins. For example, such techniques as immunofluorescence IHC can be employed to analyze only 1-4 protein markers of interest at a time. On the other hand, techniques such as Imaging Mass Cytometry™ (IMC™), electron microscopy and ion beam probing, and even some optical imaging techniques, can damage the tissue under study.

In addition, the variation in tissue samples and the substrates on which the tissue samples are disposed can adversely affect the functioning of certain analytical techniques. For example, variations in the composition of the substrate on which the tissue sample is placed can affect the detection signals in secondary ion mass spectrometry (SIMS). Further, analysis of a tissue section under investigation may lead to contamination of the analytical instrument used to analyze the tissue section. For example, the tissue section may contain salts, which can contaminate plasma sampling cones in ICP-MS (inductively coupled plasma mass spectrometry) systems. In addition, in single cell analysis, the use of ICP-MS for analysis of larger cells can be challenging as larger cells may undergo only incomplete disintegration. In laser ablation sampling, a change in composition of different tissue portions, e.g., from cell cytoplasm to cell nucleus, can result in differing ablation properties. That, in turn, can degrade the quality of imaging data. For example, in some cases, a small portion of a cell's nucleus may be dislodged and land on a different tissue spot, thereby generating a “hot spot.”

Optical imaging techniques can also present certain challenges. For example, in optical fluorescence techniques, autofluorescence of the tissue can interfere with the detection of probe tags attached to the tissue.

Conventional optical methods for single cell analysis can also suffer from certain shortcomings. By way of example, clogging is one of the problems that is encountered in cell analysis via flow and mass cytometry. Further, in analysis of cell smears, larger cells can create out-of-focus conditions for optical microscopy.

### SUMMARY

In one aspect, a method of imaging a biological sample is disclosed, which comprises coupling at least one affinity probe to the biological sample, wherein the at least one affinity probe includes at least one cleavable tag, thereby creating a probe-coupled biological sample, bringing the probe-coupled biological sample into contact with, or in proximity of, a capture surface so as to couple said at least one cleavable tag to the capture surface, and cleaving the at least one cleavable tag from the affinity probe, thereby generating a replica of the biological sample.

A variety of different types of biological samples can be employed in the practice of various embodiments. By way of example, and without limitation, the biological sample can be any of a tissue section, a cell, a cell aggregate, a cell smear, a tissue biopsy, a tissue microarray, a monolayer of adherent cells, one or more cells immobilized on a solid surface, and an array of cells captured on a chip such as Sieve Well.

The method can further comprise imaging the replica of the biological sample. In some embodiments, a plurality of different imaging modalities may be utilized to image the same replica. For example, a first imaging modality that causes no damage, or minimal damage, to the replica can be used to generate a first image of the replica and a second imaging modality can then be utilized to image the replica to generate a second image.

A variety of different imaging modalities may be employed. Some examples of such imaging modalities include, without limitation, imagining mass cytometry, mass spectrometry, MALDI, secondary ion mass spectrometry, electron microscopy, fluorescence optical microscopy, and optical imaging.

In some embodiments, the capture surface has a thickness in a range of about 10 nm to about 1000 nm.

In some embodiments, the step of cleaving the cleavable tag is performed so as to substantially retain information regarding a spatial distribution of the affinity probe in the biological sample. In some embodiments, the step of cleaving the at least one cleavable tag is performed so as to substantially retain information regarding a concentration of said affinity probe at a respective spatial location of the biological sample.

In some embodiments, the at least one cleavable tag comprises a reporter element. By way of example, and without limitation, the reporter element can be suitable for use in mass spectrometry, electron microscopy, fluorescent optical microscopy, MALDI and SIMS, all by way of example. In some embodiments, the reporter element comprises a metal. In some embodiments, the reporter element comprises a contrast agent.

In some embodiments, the capture surface comprises, without limitation, any of a gel (such as an ultrathin gel), a metal organic framework, a covalent organic framework, a nanomaterial, and porous silicon, among others.

In some embodiments, the capture surface can be a porous surface, e.g., a porous silicon surface. By way of example, and without limitation, in some embodiments, such a porous surface can exhibit a porosity of at least about 10%. In some embodiments, the porous surface can be a porous organic film or a nanomaterial. In some embodiments, the porous surface, e.g., a porous silicon surface, may be positioned over an underlying substrate, e.g., a silicon substrate.

In some embodiments, the capture surface is functionalized with at least one capture moiety that is configured to capture a plurality of different tags (i.e., tags of different types).

In some embodiments, the capture surface is substantially free of microbeads to which the tags can be attached. In some embodiments, the capture surface is a selective capture surface while in other embodiments, the capture surface is a non-selective capture surface.

In some embodiments, the capture surface is substantially free of the biological sample. In some embodiments, the capture surface is substantially free of any interference with the biological sample.

In some embodiments, the step of coupling the at least one affinity probe to the biological sample comprises staining the biological sample with a first affinity probe having a first cleavable tag to form a first replica of the biological sample, subsequently removing residual affinity probes and the associated cleavable tags, if any, from the biological sample, and, subsequent to the removing, staining the biological sample with a second affinity probe having a second cleavable tag to form a second replica of the biological sample.

In some embodiments, a first replica of a biological sample is generated using a first affinity probe having a first cleavable tag and a second replica of the biological sample is generated using a second affinity probe having a second cleavable tag (i.e., a cleavable tag different from the first cleavable tag). The two replicas can be imaged using different imaging modalities, where each imaging modality is suitable for imaging one of the tags.

In some embodiments, a single replica of a biological sample is generated, where the replica includes a plurality of different types of cleavable tags. In some embodiments, an affinity probe having a plurality of different types of cleavable tags can be employed for generating a replica of a biological sample. In some embodiments, such replicas can be imaged using different imaging modalities, where each imaging modality is suitable for imaging one type of the tags.

In some embodiments, the method comprises removing at least a portion of residual affinity probes, if any, from the biological sample subsequent to the formation of the replica and using the biological sample to generate another replica.

In a related aspect, a method of imaging a biological sample is disclosed, which comprises coupling at least one affinity probe to the biological sample, wherein the at least one affinity probe includes at least one cleavable tag, and transferring the at least one cleavable tag from the biological sample to a non-selective capture surface such that a spatial distribution of the transferred at least one cleavable tag on the non-selective capture surface substantially retains relative spatial distribution of the at least one affinity probe coupled to the biological sample.

Further understanding of various aspects of the present teachings can be obtained by reference to the following detailed description in conjunction with the associated drawings, which are described briefly below.

### BRIEF DESCRIPTION OF THE DRAWINGS

FIG. 1 is a flow chart depicting various steps in an embodiment of a method according to the present inventions for imaging a tissue sample,

FIG. 2A schematically depicts a non-selective capture surface and a tissue section stained with an affinity probe having a cleavable affinity tag,

FIG. 2B schematically depicts the stained tissue section brought in close proximity of the capture surface to facilitate the transfer of the tags from the tissue section to the capture surface,

FIG. 2C schematically shows the capture surface with the cleavable tags transferred thereto,

FIG. 3 schematically shows a cleavable tag including its associated reporter,

FIG. 4A schematically depicts a porous capture surface disposed on a porous substrate,

FIG. 4B schematically depicts cleavable tags captured by a porous capture surface,

FIG. 5 schematically depicts a plurality of stained cells disposed on a substrate that has been brought into contact with a capture surface to facilitate the formation of a replica of the stained cells, and

FIG. 6 schematically depicts a plurality of cells disposed on a substrate having a plurality of channels that facilitate the transfer of cleavable tags to a capture surface.

### DETAILED DESCRIPTION

In imaging of biological samples, it is typically desired to image multiple target analytes (parameters) of the sample, e.g., a tissue section. The problems of detecting more parameters than an imaging method allows, may be conventionally overcome by cyclic staining of the sample, e.g., using cyclic ImmunoFluorescence (CyCIF) or CODEX. As these methods rely on staining the same sample multiple times, they are incompatible with imaging methods that cause damage to the sample. For example, an IMC imaging modality can concurrently image 50 parameters associated with a biological sample. However, the process of imaging destroys the sample. Fluorescence microscopy can typically be used to image 4-6 parameters associated with a biological sample, but the imaging can be done in multiple cycles to read additional parameters, e.g., up to 100 parameters using 16-25 cycles. This method is, however, inherently slow (limited by the speed of staining) and can suffer from autofluorescence effects or repetitive staining damage from the tissue being imaged.

The present inventions are generally directed to methods for imaging biological samples in which replicas of a biological sample, e.g., a tissue section (including FFPE or frozen), can be generated and used to image target analytes, such as biomolecules of interest in the sample. A biomolecule may be classified as a protein, an oligonucleotide, a lipid, a carbohydrate, or a small molecule or combinations thereof. Alternatively, or in addition, a biomolecule may be classified by its functionality. The biomolecule is not particularly limited and can have a variety of different molecular structures and functions. For example, an oligonucleotide may be a single stranded DNA molecule, optionally cDNA that hybridizes under stringent conditions to a target nucleic acid analyte (e.g., a sample nucleic acid biomolecule) or the oligonucleotide can be an aptamer. For example, a biomolecule may be an oligonucleotide that specifically hybridizes a target oligonucleotide, such as a target mRNA endogenous to a sample (e.g., hybridizes to the sample oligonucleotide). Hybridization may be of a sequence that is more than 8, more than 10, more than 15, or more than 20 nucleotides. As noted above, in certain aspects, a biomolecule may be classified by its functionality. For example, a biomolecule may be an affinity reagent, an antigen (e.g., an analyte specifically bound by an affinity reagent), or an enzyme substrate.

As discussed in more detail below, embodiments of the methods according to the present inventions provide a number of advantages. For example, they allow the acquisition of images of the biological sample while preserving the biological sample and further allow the use of different imaging modalities for imaging the replicas.

The terms “affinity probe” and “affinity reagent” or “probe” are used herein interchangeably to refer to a compound that can specifically bind to a target molecule, e.g., a biomolecule such as a protein, or an oligonucleotide. Typically, an affinity probe forms a covalent bond with the target molecule.

By way of example, an affinity reagent may be an antibody (e.g., or fragment thereof), aptamer, receptor (e.g., or portion thereof), or any other molecule, such as a biomolecule, that specifically binds to a target (e.g., an avidin, such as streptavidin, that specifically binds biotin). For example, an affinity reagent may be associated with an antibody, may be used to detect and/or analyze the presence of its target antigen in a sample, such as the presence of a cytokine, viral protein, cancer biomarker, or the like. By way of further illustration, and without limitation, some examples of affinity probes can include, without limitation, Somamers, DARPins, affimers, avimers, knottins, monobodies, and affinity clamps.

The term “cleavable affinity tag,” or “cleavable tag,” or an “affinity tag” or a “tag,” as used herein, refers to a compound that can be part of an affinity probe and can be cleaved from the remainder of the affinity probe, e.g., via application of energy, to release the affinity tag. For example, an affinity tag can be a compound that can be coupled to a portion of an affinity probe via a cleavable chemical bond, e.g., a covalent bond that can be cleaved via application of UV radiation thereto. In such embodiments, the combination of the affinity probe and the affinity tag can be referred to as an affinity probe having a cleavable affinity tag. Some examples of suitable affinity tags can include, without limitation, tags suitable for use with various methodologies such as optical imaging, IMC, MALDI, SIMS, electron microscopy, Fluorescence, OES, and IHC. The tag may comprise any suitable structure, including polymer based, beads, particles, nanoparticles, oligonucleotides, and molecular tags.

By way of example, in some embodiments, a tag could be a chemical moiety that provides a distinguishable signal indicative of the presence of a target analyte or analyte complex with which it is associated, as for example through linkage to an affinity product that in turn recognizes the analyte or analyte complex. In some embodiments, the tag can contain an element or an isotope (or multiple copies thereof) that provide the distinguishable signal. A tag can include for example an element or isotope of an element that is associated with an analyte or analyte complex and which is measured to determine the presence of the analyte. A tag can also include, for example, any distinguishable component (e.g., an element or isotope or multiple copies thereof) that is provided on the surface or within the body of, or is otherwise associated with, a particle and serves to distinguish that particle from other particles. Further details regarding examples of suitable tags for use in various embodiments can be found, for example, in U.S. Pat. No. 10,436,698B1 and/or WO2020055813A1, each of which is herein incorporated by reference in its entirety.

The term “capture surface” as used herein refers to a material layer having a surface area that can be used to capture an affinity tag. A “non-selective capture surface” refers to a capture surface that can be used to capture a plurality of different affinity tags or other molecules of interest. A non-selective capture surface is capable of capturing multiple tags, rather than being capable of capturing only one or more specific tag(s). By way of example, a non-selective capture surface may be used to capture signaling chemicals that are excreted by a shedding cell. In another example, a non-selective capture surface may be used to capture metabolites produced when a cell is exposed to a pharmaceutical compound, e.g., to screen how cells of the same type process different pharmaceutical compounds. By way of example, the tags collected from the same cells could provide information regarding how each cell changes as a result of the exposure to a particular pharmaceutical compound. The non-selective capture surface can be such that it captures all versions of affinity tags used in experiment, but at the same time it could let salts and other potential contaminants wash away. Thus, while this behavior can be viewed as selectivity in a broader sense, the non-selective capture surface captures the tags non-discriminately and is therefore non-selective among various tags.

The term “replica” as used herein refers to a capture surface to which cleavable tags and/or native atoms/molecules available from the sample have been transferred.

The term “about” as used herein to modify a numerical value is intended to indicate a variation of at most +/−10% around that value.

The term “substantially” as used herein is intended to indicate a deviation, if any, of at most 10% relative to a complete condition or state.

The term “coupling” as used herein for indicating the relationship of two moieties, e.g., a biological sample and an affinity probe, indicates the presence of a connection between those moieties, e.g., via a covalent, a Van der Waals, or an ionic chemical bond. For example, an affinity probe can be coupled to surface protein markers of cells, e.g., cells in a tissue sample.

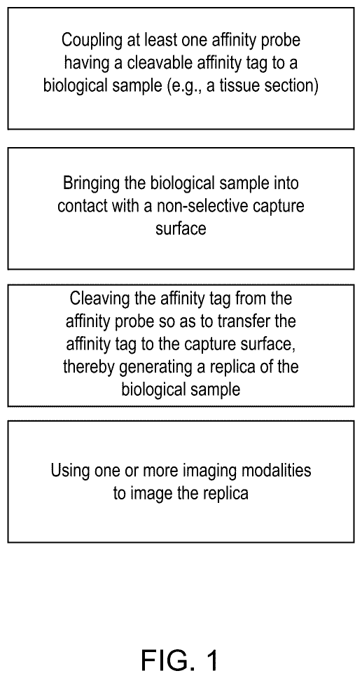

With reference to the flow chart of FIG. 1, in one embodiment of a method according to the present inventions for imaging a biological sample, an affinity probe having a cleavable affinity tag, or alternatively a native biological compound, is coupled to a biological sample (e.g., a tissue section, one or more cells, etc.). Subsequently, the biological sample is brought into contact with, or in proximity of, a non-selective capture surface and the affinity tag is cleaved from the affinity probe so as to transfer the affinity tag to the non-selective capture surface, thereby forming a replica of the biological sample. In some embodiments, the affinity probe having a cleavable affinity tag is brought into contact with the capture surface via the affinity tag, and subsequently the affinity probe is cleaved. In other embodiments, the affinity probe having a cleavable affinity tag is brought in proximity of the capture surface, and subsequently the affinity probe is cleaved, and subsequently the affinity tag contacts the capture surface. In some cases the movement of the tags to the capture surface can be motivated by electric forces, or by gravity or centripetal force, or by diffusion or by liquid flow. In the case of using a native biological compound, the compound is removed from the replica in a similar fashion. The replica of the biological sample can then be imaged. As discussed below, in many embodiments, a variety of different imaging modalities can be employed for imaging the replica. Replicas of cell smears can also be used for single cell analysis in suspension.

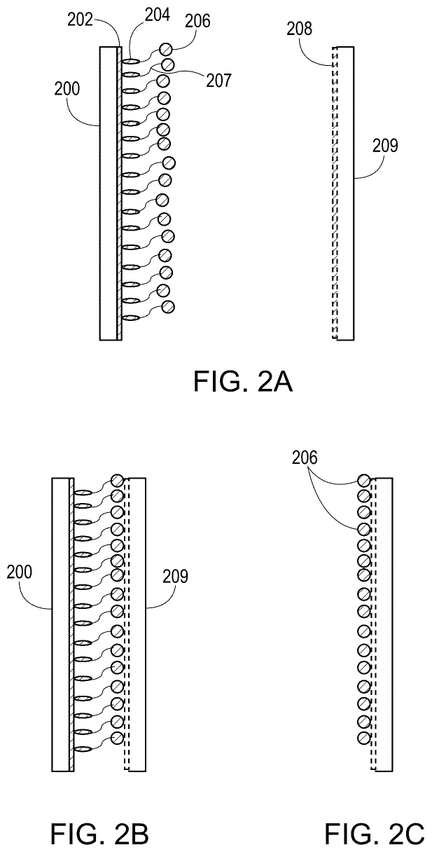

By way of further illustration, FIG. 2A schematically depicts a support substrate 200 on which a tissue section 202 is disposed. The tissue section is stained with an affinity probe 204 that includes a cleavable affinity tag 206. More specifically, in this embodiment, the affinity tag is coupled (bonded) to the remainder of the affinity probe via a cleavable chemical bond 207, e.g., a covalent bond that can be broken via application of UV radiation thereto. A variety of staining techniques can be employed in the practice of the present teachings. By way of example, the staining step can be in the form of immune-staining or in the form of DNA/RNA hybridization. Some examples of affinity probes are mentioned above. In some embodiments, cleavable bonds can be activated by light (e.g., UV), or chemically (e.g., enzymatically), electrochemically or by heat. In some embodiments, molecules of interest may be extracted from a biological sample by liquid extraction, by electrically instigated motion (e.g., electrophoresis), by osmotic effect, or by high gravitational force (e.g., centrifugal force), among others.

FIG. 2A also schematically depicts a non-selective capture surface 208 that is disposed on an underlying substrate 209. As discussed in more detail below, the cleavable tags can be transferred from the stained tissue section to the capture surface as seen in FIG. 2B. The resulting effect is shown in FIG. 2C, where the affinity tags 206 are all cleaved and attached onto substrate 209.

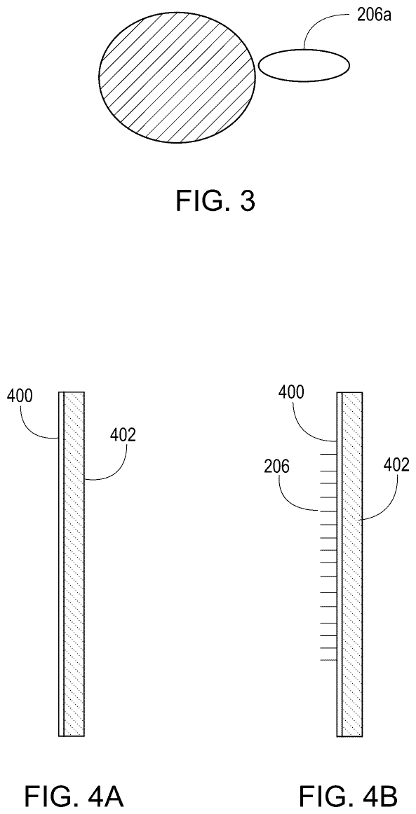

With reference to FIG. 3, the cleavable affinity tag 206 can contain a reporter 206a that can be interrogated using one or more imaging modalities. In general, a variety of imaging modalities can be employed for the detection of the cleavable tag (e.g., the reporter of the cleavable tag). By way of example, in some embodiments, the reporter of the cleavable tag can be a metal element, such as osmium and a lanthanide. In some embodiments, the reporter of the cleavable tag can be a fluorescent dye or a contrast agent for electron microscopy. Alternatively, one could use an MRI contrast reagent, a radioactive contrast reagent, an X-ray analysis contrast reagent, a molecule suitable for detection by organic mass spectrometry such as SIMS or MALDI or ESI. Moreover, organic mass spectroscopy can read the reporters and the native molecules simultaneously. Imaging mass spectroscopy, however, would only read native elements and tags but may damage or destroy native molecules in the process.

As shown schematically in FIGS. 2A and 2B, the support substrate 200 can be brought into proximity of the non-selective capture surface 208 (herein referred to for simplicity as the capture surface 208) such that the cleavable tags 206 associated with the affinity reagent are in contact with, or in proximity of, the capture surface.

Subsequently, the cleavable tags can be cleaved and transferred to the capture surface, as shown schematically in FIG. 2C. The cleavage of the tags can be achieved using a variety of different energy modalities. By way of example, the cleavage of the tags can be accomplished using photo-cleavage, chemical cleavage, electrochemical cleavage, among others.

In some embodiments, the transfer of the cleaved tags to the capture surface can be facilitated using electric forces, e.g., in a manner similar to the way electric forces are employed in electrophoresis, or via diffusion, or via flow of a liquid, as discussed in more detail below. By way of example, in some embodiments in which the capture surface and an underlying substrate on which the capture surface is disposed are formed of porous materials, the transfer of the cleaved tags can be accomplished via the flow of a liquid through the biological sample of interest (e.g., a tissue section), and then through the porous capture surface followed by its outflow through the channels of the underlying porous substrate. In some embodiments, the transfer of the cleavable tags to the capture surface can be facilitated via an artificial gravity created in a centrifuge, via magnetic forces or other suitable forces.

The capture surface can be any suitable surface that can retain the tags that are transferred from the biological sample, which in this embodiment corresponds to the stained tissue section. As noted above, the capture surface can allow the capture of a plurality of different tags. Some examples of suitable capture surfaces can include, without limitation, an ultrathin gel, a metal-organic framework, a covalent organic framework, nanomaterials, porous silicon, among others. The use of an organic capture surface can be particularly advantageous in applications in which ICP-MS is utilized for interrogating the capture surface subsequent to the transfer of the cleavable tags thereto, e.g., by facilitating laser ablation as well as material disintegration in an ICP ion source. By way of example, in some embodiments, the capture surface can be a covalent organic framework. As known in the art, covalent organic frameworks are a class of crystalline porous organic polymers that exhibit permanent porosity and highly ordered structures. Some examples of covalent organic frameworks and methods for their synthesis are disclosed in an article entitled “Covalent Organic Frameworks: Design, Synthesis, and Function” by Geng et al. published in Chem Rev. 2020 Aug. 26:120(16 ): 8814-8933, which is herein incorporated by reference in its entirety.

In some embodiments, the capture surface can be a porous surface. By way of example, FIG. 4A schematically depicts a porous capture surface 400 that is supported on an underlying substrate 402. FIG. 4B schematically depicts the capture surface 400 with a plurality of tags 206 attached thereto. The transfer of the tags 206 to the capture surface can be achieved, for example, in a manner discussed above in connection with FIGS. 2A, 2B, and 2C. In the embodiment of FIGS. 4A and 4B, similar to the capture surface 400, the underlying substrate 402 is also porous. In some embodiments, the porous capture surface 400 can exhibit a porosity in a range of about 10% to about 99%. In some embodiments, the porous capture surface can increase the upper limit of the dynamic range for capturing the tags via the capture surface by up to 1000×. A relevant measure is to compare the surface area of a spot of a particular diameter for the porous surface versus a flat surface, By making the surface porous, one can increase available surface area by up to 1000×. Accordingly, the ratio roughly translates to the gain in the number of tags that can be captured versus the flat non-porous surface. By way of example, and without limitation, the porous capture surface 400 can be in the form of a porous silicon layer. By way of example, the porous silicon layer can be formed in a silicon substrate, e.g., via chemical etching. In other embodiments, the porous capture surface 400 can be formed of a porous organic film (e.g., an organic framework).

In some embodiments, the porosity of the capture surface 400 can facilitate capture and retention of the cleaved tags. Further, in some embodiments, the combination of the porous capture surface and a porous underlying substrate can facilitate the transfer of the cleaved tags from the tissue section to the capture surface via flow of a liquid (e.g., deionized water) through the tissue section and the porous capture surface. In some such embodiments, the tags can be retained within the pores of the porous capture surface and also the tags can be retained in the channels of the substrate.

Referring again to FIGS. 2A and 2B, in this embodiment, the capture surface is substantially free of moieties that would exhibit specific binding to the cleavable tags. By way of example, in embodiments, the capture surface can be substantially free of microbeads having moieties that exhibit specific binding to a single tag (i.e., they have been functionalized to capture a specific tag). In other words, in such embodiments, the indiscriminate capture of the cleavable tags by the capture surface is accomplished as a result of the material composition and/or the geometry of the capture surface.

In other embodiments, the capture surface can include moieties that can bind to multiple cleavable tags. In some cases, the capture surface can include a single moiety that can bind to multiple cleavable tags and can hence facilitate the retention of the cleavable tags by the capture surface. Some examples of such moieties can include, without limitation, surface modifications of covalent organic frameworks as discussed in the article entitled “Covalent Organic Frameworks: Design, Synthesis, and Function” by Geng et al. referenced above.

The imaging of the capture surface subsequent to the transfer of the cleavable tags thereto can be achieved using a variety of different imaging modalities suitable for the particular tags. Some examples of such imaging modalities can include, without limitation, fluorescence imaging, imaging mass cytometry, SIMS, and electron microscopy, among others.

In some embodiments, a single type of tag is used for generating the replica. In some such embodiments, such a tag can be interrogated (imaged) via different imaging modalities. By way of example, a tag carrying an osmium reporter can be imaged via both electron microscopy and IMC. Certain lanthanoid tags can be imaged via both fluorescence imaging and IMC.

The ability to image a tag via a plurality of different imaging modalities can provide certain advantages. For example, if it is discovered that one imaging modality may cause damage to the replica, another imaging modality that would result only in minor damage, or no damage, may be preferentially used as a first method for imaging the replica. Alternatively, a given imaging modality can only record specific information from a subset of the tags while another imaging modality can record different information from another subset of the tags. For instance, electron microscopy can be used for reading osmium as a tag at high spatial resolution while a subsequent IMC can be used for reading all the metal tags including osmium as independent reporters but with reduced spatial information.

The material composition of the capture surface can be designed to facilitate the use of a particular imaging modality for imaging the replica. For example, the material of the capture surface can facilitate an efficient ablation at a chosen laser wavelength when ICP-MS is employed as the imaging modality. In some embodiments, the use of an organic capture surface can facilitate ablation and ionization when imaging the surface via MALDI, where the organic surface itself may function as a matrix removing the need to apply MALDI matrix to the sample.

In some embodiments, a biological sample of interest (e.g., a tissue section) can be stained with affinity reagents having cleavable tags where the cleavable tags can be activated for breakage by a plurality of different methods. For example, one type of the cleavable tags may be activated for breakage using one kind of energy modality and another type of the cleavable tags may be activated for breakage using another kind of energy modality. This allows generating multiple replicas from the same biological sample (e.g., a tissue section). Alternatively, the first replica can be made using a first set of tags and the tissue can be stained with the second set of tags. The second set of tags can be cleaved off, for example, by the same method but this time around the tissue will be in contact with the second capture surface. This will create a second replica. The process might be repeated more than two times using the same cleaving method for tags (e.g., UV photo-cleavage).

For example, a first replica can be formed via cleavage and transfer of one kind of tags from the biological sample to a capture surface using one kind of energy modality and a second replica can be formed via cleavage and transfer of another kind of tags from the biological sample to another capture surface using another kind of energy modality. Different energy modalities are not needed if the sample is not stained all at once. In some embodiments, if the biological sample is stained twice then after the first staining the first replica is created and after the second staining the second replica is created. The process can be repeated based on the number of different tags coupled to the biological sample. In this manner, a biological sample (e.g., a tissue section) can be stained once but can be used to create more than one replica thereof.

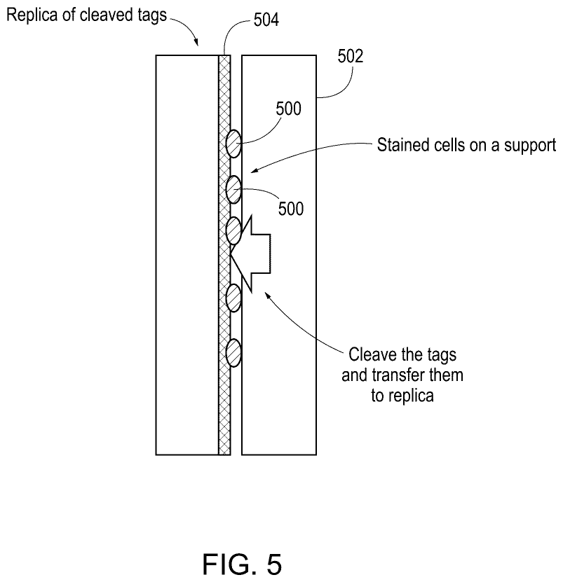

The present inventions are not limited to imaging tissue sections, but rather, they can be used to image a variety of biological samples. By way of example, with reference to FIG. 5, the present inventions can be employed to generate a replica of a plurality of cells 500 disposed on an underlying support substrate 502. Similar to the previous embodiment, the cells 500 can be stained with an affinity probe (not shown), such as the affinity probes discussed above, having a cleavable tag and the cleavable tags can be transferred to a non-selective capture surface 504 in a manner discussed above, e.g., via bringing the cells in contact with, or in proximity of, the capture surface and causing the cleavage of the tags, thereby generating the replica.

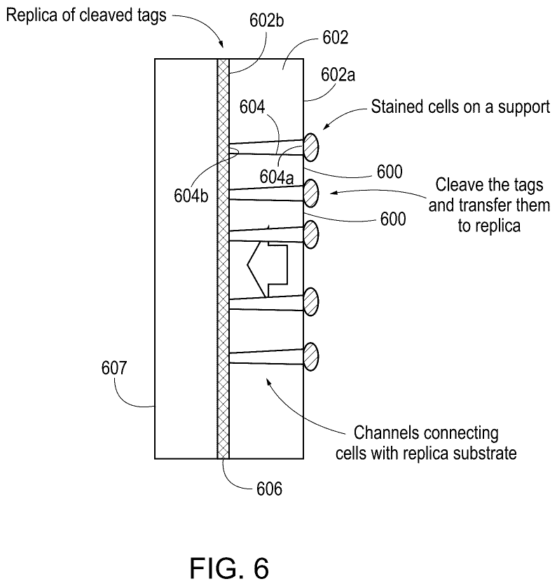

FIG. 6 schematically depicts an embodiment in which a plurality of cells 600 are disposed on a substrate 602 having two opposed surfaces 602a/602b with a plurality of channels 604 that extend between two sets of openings 604a/604b, formed on the surfaces 602a/602b, respectively. This is an example of probing rather than imaging of the tags. Each spot corresponds to a single cell and gets recorded (probed) as a single record of many tags. Moreover, the cells in this example can be maintained live for further experiments. The cells 600 are disposed on the surface 602a so as to be in substantial register with the openings 604a. The cells are stained with an affinity probe (not shown) having a cleavable affinity tag (not shown), such as those discussed above. A replica of the cells can be generated by bringing the surface 602b into contact with a non-selective capture surface 606, which is disposed on an underlying substrate 607. The cleavable tags can then be cleaved via application of energy thereto (such as UV radiation), e.g., in a manner discussed above, and they can be transferred through the channels 604 to the capture surface.

Embodiments of a replica of a biological sample according to the present teachings can provide several advantages. For example, and without limitation, the replicas can be used to image a biological sample without causing any damage thereto. This in turn allows imaging the same biological sample multiple times, e.g., using a plurality of different stains and/or imaging modalities. By way of example, multiple cycles of an IMC imaging modality can be used to image, in each cycle, 50 parameters (e.g., 50 target analytes of interest) and the cycles can be repeated, for example, four times using 4 replicas to image 200 parameters of the same biological sample (e.g., a tissue section).

As noted above, the present inventions allow imaging a biological sample via a plurality of different imaging modalities. For example, two replicas of a biological sample can be formed using different tags and each replica can be imaged using a different imaging modality suitable for a particular tag. In another embodiment, a replica of a biological sample can be formed using a plurality of different types of tags, where each tag can be imaged via a different imaging modality. In other words, a single replica can be formed with different types of tags, where each type can be interrogated (imaged) via a different imaging modality.

In some embodiments, a biological sample can be stained with an affinity probe having a first cleavable tag and the stained biological sample can be used to form a first replica, which can be imaged via a first imaging modality. Subsequently, the biological sample can be cleaned (e.g., via washing or otherwise) from residuals of the affinity probe and/or the tags. The cleaned biological sample can then be re-stained with a different type of tag (which can be imaged using a different imaging modality), using the same type of affinity probe cleavably attached to the new tags or a different affinity probe cleavably attached to the new tags, and a replica based on the re-stained biological sample can be formed. Each replica can be imaged with a different imaging modality suitable for imaging the different tags. In other embodiments, the affinity probe can substantially cease to have affinity probe activity upon cleaving of the cleavable tag from the affinity probe (for example a biological or chemical trigger both cleaves the cleavable tag and deactivates the affinity probe), such that residuals of the affinity probe are substantially removed from the biological sample.

In addition, the use of replicas can lead to less contamination of an imaging instrument. For example, as noted above, tissue can contain salts and excessive organic frameworks that can contaminate plasma sampling cones in ICP-MS systems. In contrast, in embodiments, replicas can contain only molecules digestible by the plasma. This means that the framework material could be chosen to fully evaporate in an ICP ion source when the cell material sometimes still makes it to the interface cones and sticks there.

Further, the replicas can be formed such that the capture surface is substantially uniform, which can in turn render the imaging process more predictable. For example, in some embodiments, the use of a substantially uniform and thin replica (e.g., a replica having a thickness in a range of about 10 nm to about 1000 nm) can essentially eliminate the generation of “hot spots,” which can occur, e.g., when using laser ablation to image a tissue section, as discussed above.

In addition, the thickness of replicas generated according to the present teachings can be much less than the thickness of the respective biological sample, e.g., a tissue section. The smaller thickness of the replicas can result in less material that needs to be removed (e.g., via ablation or ion beam imaging).

Further, the use of replicas according to the present inventions with optical imaging techniques can also provide certain advantages. For example, it can eliminate the problem of the interference of autofluorescence (which can be different for different tissue samples) with the detection of tags. With replicas, the type of tissue from which a replica is formed does not change the autofluorescence. Moreover, the autofluorescence of replicas can be controlled via selection of the material of the capture surface.

Further, in embodiments, the use of replicas for imaging biological samples can also solve certain problems associated with single cell analysis by flow cytometry and mass cytometry. For example, the use of replicas for imaging cell smears can solve the problem of clogging of the system which sometimes occurs when liquid samples are analyzed. Further, the use of replicas according to the present inventions can solve certain problems associated with the imaging or recording of tag signals of large cells. As noted above, large cells can create out of focus conditions for optical microscopy and cause incomplete disintegration for ICP-MS based analysis. In some embodiments, such problems associated with imaging large cells can be eliminated by generating thin replicas of the large cells. In some embodiments, a replica with the same thickness can be used for imaging large and small cells.

Those having ordinary skill in the art will appreciate that various changes can be made to the above embodiments without departing from the scope of the present teachings.

1. A method of imaging a biological sample, comprising:
coupling at least one affinity probe to the biological sample, wherein the at least one affinity probe includes at least one cleavable tag, thereby creating a probe-coupled biological sample,
bringing the probe-coupled biological sample into contact with, or in proximity of, a capture surface so as to couple said at least one cleavable tag to the capture surface, and
cleaving the at least one cleavable tag from the affinity probe, thereby generating a replica of the biological sample.

2. The method of claim 1, wherein the biological sample comprises any of a tissue section, a cell, a cell aggregate, a cell smear, a tissue biopsy, a tissue microarray, a monolayer of adherent cells, one or more cells immobilized on a solid surface, and an array of cells captured on a chip.

3. The method of claim 1, further comprising imaging said replica of the biological sample.

4. The method of claim 3, wherein said step of imaging the replica of the biological sample comprises utilizing a plurality of different imaging modalities to image the replica.

5. The method of claim 4, wherein said imaging modalities comprise any of imaging mass cytometry, mass spectrometry, MALDI, secondary ion mass spectrometry, electron microscopy, fluorescence optical microscopy, SIMS and optical imaging.

6. The method of claim 1, wherein said capture surface has a thickness in a range of about 10 nm to about 1000 nm.

7. The method of claim 1, wherein the step of cleaving the cleavable tag is performed so as to substantially retain information regarding a spatial distribution of said affinity probe in said biological sample.

8. The method of claim 2, wherein the step of cleaving the at least one cleavable tag is performed so as to substantially retain information regarding a concentration of said affinity probe at a respective spatial location of the biological sample.

9. The method of claim 1, wherein the at least one cleavable tag comprises a reporter element.

10. (canceled)

11. The method of claim 9, wherein said reporter element comprises any of a metal and a contrast agent.

12. The method of claim 1, wherein said capture surface comprises a porous surface.

13. The method of claim 12, wherein said porous surface exhibits a porosity of at least about 10%.

14. (canceled)

15. The method of claim 12, wherein said porous surface comprises a porous silicon surface positioned over an underlying silicon substrate.

16. (canceled)

17. The method of claim 1, wherein said capture surface comprises any of an ultrathin gel, a metal-organic framework, a covalent organic framework, a nanomaterial, a porous organic film, and porous silicon.

18. The method of claim 1, wherein said capture surface is functionalized with at least one capture moiety that is configured to capture a plurality of different tags.

19. The method of claim 1, wherein said capture surface is substantially free of microbeads to which the tags can be attached.

20. The method of claim 19, wherein said capture surface is a selective capture surface.

21. (canceled)

22. The method of claim 1, wherein the step of coupling at least one affinity probe to the biological sample comprises staining the biological sample with a first affinity probe having a first cleavable tag to form a first replica of the biological sample, removing at least a portion of said first affinity probe from the biological sample, and, subsequent to the removing, staining the biological sample with a second affinity probe having a second cleavable tag to form a second replica.

23. (canceled)

24. The method of claim 1, wherein said at least one replica of the biological sample comprises a first replica generated using a first affinity probe having a first cleavable tag and a second replica generated using a second affinity probe having a second cleavable tag, and further comprising imaging said first replica using a first imaging modality and imaging said second replica using a second different imaging modality.

25. The method of claim 1, wherein said at least one affinity probe comprises a plurality of different types of cleavable tags.

26. (canceled)

27. The method of claim 25, further comprising imaging the replica using a plurality of imaging modalities, wherein each imaging modality is used to image a different type of said tags.

28. The method of claim 1, further comprising removing at least a portion of residual affinity probes, if any, from the biological sample subsequent to formation of the replica and using the biological sample to generate another replica.

29. A method of imaging a biological sample, comprising:
coupling at least one affinity probe to the biological sample, wherein the at least one affinity probe includes at least one cleavable tag,
transferring the at least one cleavable tag from the biological sample to a non-selective capture surface such that a spatial distribution of the transferred at least one cleavable tag on the non-selective capture surface substantially retains relative spatial distribution of the at least one affinity probe coupled to the biological sample.
